(19) United States
(12) Patent Application Publication
(10) Pub. No.: US 2019/0350671 A1
(43) Pub. Date: Nov. 21, 2019

(54) AUGMENTED REALITY CATHETER TRACKING AND VISUALIZATION METHODS AND SYSTEMS

(71) Applicants: UNIVERSITY OF MARYLAND, COLLEGE PARK, College Park, MD (US); UNIVERSITY OF MARYLAND, BALTIMORE, Baltimore, MD (US)

(72) Inventors: Amitabh VARSHNEY, Potomac, MD (US); Xuetong SUN, College Park, MD (US); Sarah MURTHI, Lutherville, MD (US); Gary SCHWARTZBAUER, Glenwood, MD (US)

(21) Appl. No.: 16/418,531

(22) Filed: May 21, 2019

Related U.S. Application Data
(60) Provisional application No. 62/674,134, filed on May 21, 2018.

(51) Int. Cl. A61B 90/00; A61M 25/01; A61B 34/20

(52) U.S. Cl. CPC A61B 90/36; A61M 25/0105; A61B 2034/102; A61B 2090/365; A61M 2025/0166; A61B 34/20

(57) ABSTRACT

Systems, methods, apparatuses, and computer program products for managing building energy utilization are provided. One method may include detecting movement of the catheter as it is being inserted into an object, and calculating a location of an area of the catheter that is embedded in the object. The method may also include generating a virtual image of the embedded portion of the catheter based on the detected movement of the catheter and location of the area of the catheter that is embedded. The method may further include transmitting the virtual image of the embedded area of the catheter to a display unit. Further, the method may include overlaying the virtual image in a user's field of view in the display unit to mimic the position of the entire catheter including the area embedded in the object.

### CROSS-REFERENCE TO RELATED APPLICATIONS

This application claims priority from U.S. provisional patent application No. 62/674,134 filed on May 21, 2018. The contents of this earlier filed application are hereby incorporated in their entirety.

### FIELD

Some example embodiments may generally relate to augmented reality guided catheters. More specifically, certain example embodiments may relate to methods, apparatuses and/or systems for augmented reality catheter tracking and visualization.

### BACKGROUND

Extra ventricular drainage (EVD) is a high-risk medical procedure that involves inserting a catheter inside a patient's skull. The catheter is inserted through the brain and into the ventricle to drain cerebrospinal fluid relieving elevated intracranial pressure. Once the catheter has entered the skull, its tip can no longer be seen or tracked using conventional technology. The neurosurgeon has to imagine its location inside the cranium, and direct the catheter towards the ventricle using only anatomic landmarks. The EVD catheter may be thin and therefore difficult to track using infra-red depth sensors. In addition, traditional optical tracking using fiducial or other markers inevitably changes the shape or weight of the medical instrument.

In general, proper catheter placement is essential to the success of an EVD procedure. To accomplish this, a detailed preoperative medical image has been overlayed on intraoperative images. Further, tracking of the imaging probe has been attempted. Even so, conventional EVD procedures make it challenging to detect and track with commodity depth sensors. Thus, there is a need in the art for an EVD catheter having a distal tip that is trackable by an operator. There is also a need of being able to visualize the location of the catheter inside the patient, and provide an optical marker and tracking technique suitable for augmented reality application.

### SUMMARY

One embodiment is directed to a method for visualization and tracking a catheter. The method may include detecting movement of the catheter as it is being inserted into an object. The method may also include calculating a location of an area of the catheter that is embedded in the object. The method may further include generating a virtual image of the embedded portion of the catheter based on the detected movement of the catheter and location of the area of the catheter that is embedded. In addition, the method may include transmitting the virtual image of the embedded area of the catheter to a display unit. Further, the method may include overlaying the virtual image in a user's field of view in the display unit to mimic the position of the entire catheter including the area embedded in the object.

Another embodiment is directed to an apparatus for visualizing and tracking a catheter. The apparatus may include at least one processor and at least one memory comprising computer program code. The at least one memory and computer program code may be configured, with the at least one processor, to cause the apparatus at least to detect movement of a catheter as it is being inserted into an object. The apparatus may also be caused to calculate a location of an area of the catheter that is embedded in the object. The apparatus may further be caused to generate a virtual image of the embedded portion of the catheter based on the detected movement of the catheter and location of the area of the catheter that is embedded. In addition, the apparatus may be caused to transmit the virtual image of the embedded area of the catheter to a display unit. Further, the apparatus may be caused to overlay the virtual image in a user's field of view in the display unit to mimic the position of the entire catheter including the area embedded in the object.

Another embodiment is directed to a system for visualizing and tracking a catheter. The system may include sensing means for detecting movement of a catheter as it is being inserted into an object. The system may also include processing means for calculating, based on information obtain from the sensing means, a location of an area of the catheter that is embedded in the object. The system may further include generating means for generating a virtual image of the embedded portion of the catheter based on the detected movement of the catheter and the location of the area of the area of the catheter that is embedded in the object. In addition, the system may include transmitting means for transmitting the virtual image of the embedded area of the catheter to a display unit. Further, the system may include displaying means for overlaying the virtual image in a user's field of view in the display unit to mimic the position of the entire catheter including the area embedded in the object.

Another embodiment is directed to computer readable medium comprising program instructions stored thereon for performing a method. The method may include detecting movement of a catheter as it is being inserted into an object. The method may also include calculating a location of an area of the catheter that is embedded in the object. The method may further include generating a virtual image of the embedded portion of the catheter based on the detected movement of the catheter and location of the area of the catheter that is embedded. In addition, the method may include transmitting the virtual image of the embedded area of the catheter to a display unit. Further, the method may include overlaying the virtual image in a user's field of view in the display unit to mimic the position of the entire catheter including the area embedded in the object.

### BRIEF DESCRIPTION OF THE DRAWINGS

For proper understanding of example embodiments, reference should be made to the accompanying drawings, wherein:

FIG. 1 illustrates a configuration of a system, according to an example embodiment.

FIG. 2 illustrates a workflow of the system in FIG. 1, according to an example embodiment.

FIG. 3 illustrates a view seen through a head-mounted display (HMD), according to an example embodiment.

FIG. 4 illustrates a reference system mapping process, according to an example embodiment.

FIG. 5 illustrates a catheter with markings, according to an example embodiment.

FIG. 6 illustrates a special representation of the catheter on an image plane, according to an example embodiment.

FIG. 7 illustrates a position of the catheter in a camera space, according to an example embodiment.

FIGS. 8(a)-8(h) illustrate a procedure for detecting endpoints of the color bands, according to an example embodiment.

FIG. 9(a) illustrates a setup for testing the tracking accuracy over a grid, according to an example embodiment.

FIG. 9(b) illustrates a setup for testing tracking accuracy with a third party tracker, according to an example embodiment.

FIG. 10 illustrates an accuracy tracking approach over the grid, according to an example embodiment.

FIG. 11(a) illustrates the catheter from a camera and the third party tracker just after calibration with a calculated catheter position in a camera space and a virtual catheter position in the HMD, according to an example embodiment.

FIG. 11(b) illustrates the catheter from a camera and the third party tracker with an unsteady alignment, according to an example embodiment.

FIG. 12(a) illustrates a distribution of the distances between a catheter's tip location and the third party tracker, according to an example embodiment.

FIG. 12(b) illustrates another distribution of an angle formed by the catheter orientations and the third party tracker, according to an example embodiment.

FIG. 13 illustrates a plot of recorded distances, according to an example embodiment.

FIG. 14 illustrates various components of the system, according to an example embodiment.

FIG. 15 illustrates a flow diagram of a method, according to an example embodiment.

FIG. 16(a) illustrates an example block diagram of an apparatus, according to an embodiment.

FIG. 16(b) illustrates an example block diagram of another apparatus, according to an example embodiment

FIG. 17(a) illustrates implementation of the system in a mock-up surgical environment, according to an example embodiment.

FIG. 17(b) illustrates the system used in a mock-up surgical environment with a catheter shown without interference, according to an example embodiment.

### DETAILED DESCRIPTION

It will be readily understood that the components of certain example embodiments, as generally described and illustrated in the figures herein, may be arranged and designed in a wide variety of different configurations. Thus, the following detailed description of some example embodiments of systems, methods, apparatuses, and computer program products for managing building energy utilization, is not intended to limit the scope of certain embodiments but is representative of selected example embodiments.

The features, structures, or characteristics of example embodiments described throughout this specification may be combined in any suitable manner in one or more example embodiments. For example, the usage of the phrases “certain example embodiments,” “some example embodiments,” or other similar language, throughout this specification refers to the fact that a particular feature, structure, or characteristic described in connection with an embodiment may be included in at least one embodiment. Thus, appearances of the phrases “in certain example embodiments,” “in some example embodiments,” “in other example embodiments,” or other similar language, throughout this specification do not necessarily all refer to the same group of embodiments, and the described features, structures, or characteristics may be combined in any suitable manner in one or more example embodiments.

Additionally, if desired, the different functions or steps discussed below may be performed in a different order and/or concurrently with each other. Furthermore, if desired, one or more of the described functions or steps may be optional or may be combined. As such, the following description should be considered as merely illustrative of the principles and teachings of certain example embodiments, and not in limitation thereof.

Certain example embodiments may provide techniques for methods and systems for augmented reality guided catheters. For example, certain embodiments may be described in the context of an extra ventricular drainage (EVD) catheter. However, certain example embodiments may be used in conjunction with other conventional medical catheters. Other example embodiments may also be used in other fields wherein a thin elongated member must be tracked in an otherwise untraceable environment.

According to certain example embodiments, an optical marker and tracking technique suitable for augmented reality may be provided. For example, a portion of the catheter may be labeled with three distinct colors that may be detected through the implementation of an algorithm. Detection of the color bands may then be used to calculate the position of the catheter. This way, even if the tip of the needle is occluded, it may still be possible to know where it is and visualize the needle, as long as enough of the colored portion remains visible. In an example embodiment, at least two consecutive color bands must be visible in order to know where the tip of the needle is and to visualize the needle.

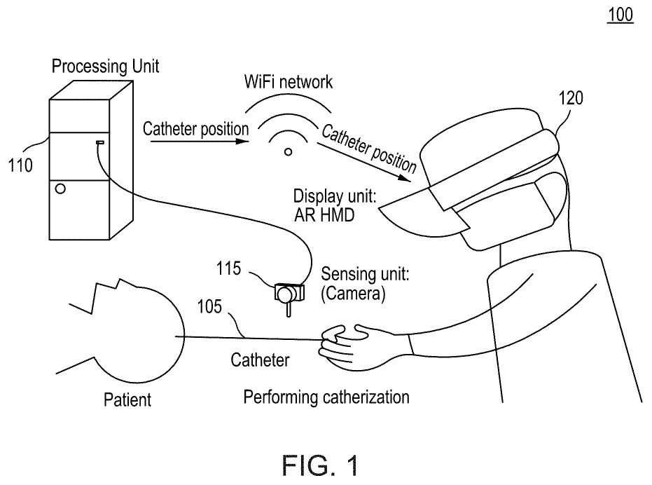

FIG. 1 illustrates a configuration of a system 100, according to an example embodiment. In an example embodiment, the system 100 may represent an augmented reality guided catheter system. As illustrated in FIG. 1, the system 100 may include a catheter 105, a processing unit 110, a sensing unit 115, and a display unit 120. In an example embodiment, the sensing unit 115 may be configured to detect the movement and position of the catheter 105 while it is being used. Further, the display unit 120 may be a head-mounted display unit (HMD), or it may be an external monitor or projection that is viewable by an operator wearing the HMD. In addition, the catheter 105 may be an existing catheter on the market, or it may be specifically configured for use with the system described herein. In other example embodiments, the catheter 105 may be a handheld device that is controlled directly by the operator. In further example embodiments, the catheter 105 may be controlled via a voice command, via a mechanical device, or by a combination thereof.

According to an example embodiment, the sensing unit 115 may be in communication with the processing unit 110, and the processing unit 110 may be in communication with the display unit 120. In addition, communication between the sensing unit 115 and the processing unit 110 may be performed via a wireless signal or via hardwired connection.

In an example embodiment, the catheter 105 may be marked with three or more patterns or colors to enable position tracking. According to certain example embodiments, the colors may be distinct from each other so that the Euclidean distance in the RGB color space of any two colors is larger than a predefined threshold. In certain example embodiments, the three colors may include red, green, and blue. However, in other example embodiments, different markers on the catheter may be used in lieu of patterns or colors. For example, instead of color bands, it may be possible to use a sheet of paper attached to the catheter on which a 2D QR code is printed. Another example may include several (e.g., 3 to 10) infrared reflective spheres attached to the catheter. In addition, the sensing unit 115 may be set in a station position. According to an example embodiment, the sensing unit 115 may be a camera, however, other sensing devices such as an infrared camera or depth sensor may also be used. Furthermore, in an example embodiment, the processing unit 110 may be configured to calculate the location of the area of the catheter that is embedded in the patient and out of view of the sensing unit 115. The processing unit 110 may also be configured to transmit an image of the embedded portion of the catheter to the display unit 120 as an overlaid image on the patient. In an example embodiment, the image data transmitted from the processing unit 110 to the display unit 120 may be done wirelessly or via a hardwired connection.

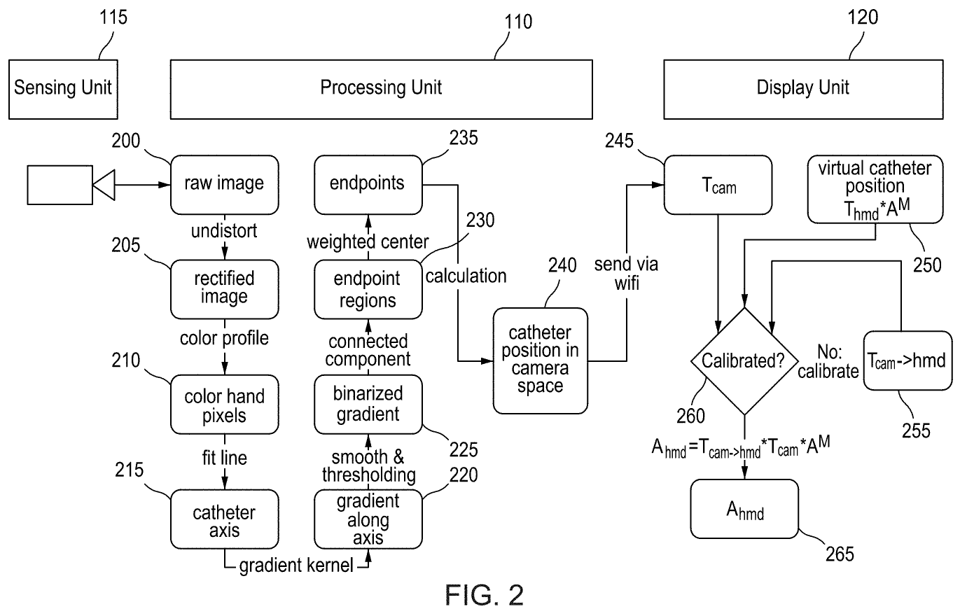

FIG. 2 illustrates a workflow of the system in FIG. 1, according to an example embodiment. As illustrated in FIG. 2, at 200, the processing unit 110 may receive from the sensing unit 115, a raw image of the catheter 105. The raw image received at the processing unit 110 may be undistorted. As such, at 205, the raw image may be rectified. After the raw image is rectified, a predefined color profile may be used to distinguish the colors of the color bands, after which at 210, color band pixels may be provided. Once color band pixels have been provided, a line may be fitted based on the color band pixels, and at 215, a catheter axis may be generated and a gradient kernel applied. At 220, a gradient along the catheter axis is generated. The gradient along the axis may be smoothed and thresholds may be applied so that at 225, a binarized gradient may be calculated.

As further illustrated in FIG. 2, after calculating the binarized gradient, connected components of the binarized image may be calculated. Then, at 230, endpoint regions of the catheter may be determined, and at 235, weighted centers of the connected components may be determined as the endpoints of the color bands on the catheter. At 240, calculations may be performed to determine the catheter position in the camera space. Information regarding the catheter position may then be sent via wireless transmission or a hardwired connection to the display unit 120. The catheter position information may be received at 245 at a certain frame T_{cam}.

At 250, the display unit 120 may receive information regarding the virtual catheter position in the HMD space T_{hmd}*A^{M}, where A^{M} represents the virtual catheter's coordinate in its own model space. At 255, the display unit 120 may determine T_{cam->hmd}, which is the transformation that transforms coordinates in the camera space to coordinates in the HMD space. As illustrated in FIG. 2, at 255, a calibration process may be performed, which may need to be performed once, or whenever the user thinks recalibration is needed. Further, at 260, the display unit 120 may receive information regarding the camera frame T_{cam}, virtual catheter position in its model space A^{M}, and the transformation T_{cam->hmd} if calibration has been performed in 255. If calibration has been performed, then, at 265, the coordinate of the virtual catheter in the HMD space A_{hmd} may be determined. As illustrated in FIG. 2, A_{hmd} may be determined by the equation: A_{hmd}=T_{cam->hmd}*T_{cam}*A^{N}.

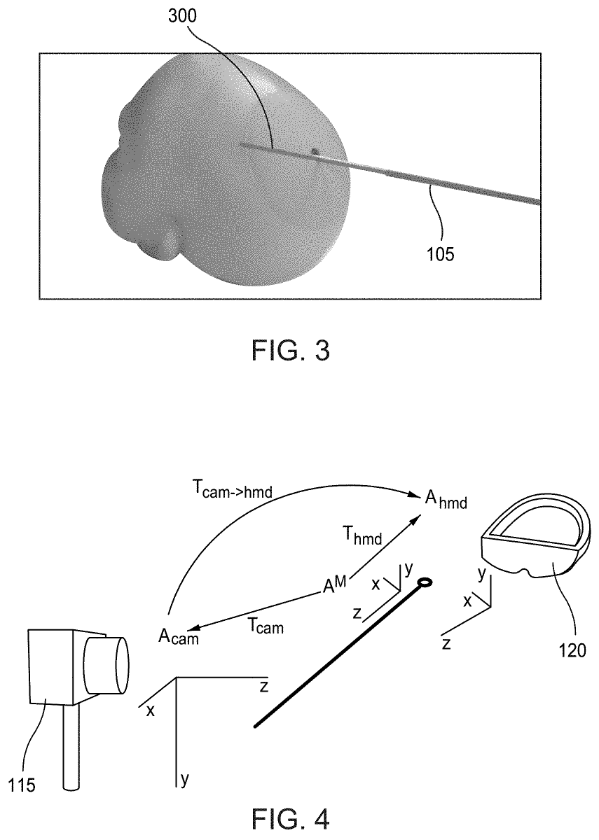

FIG. 3 illustrates a view seen through an HMD, according to an example embodiment. As illustrated in FIG. 3, the catheter 105 may be tracked by the sensor unit 115, such as a webcam or similar type camera. In addition, according to an example embodiment, a virtual representation of the catheter 105 may be rendered to overlay the real catheter. For example, as illustrated in FIG. 3, the occluded part 300 of the catheter 105 may also be displayed with the rest of the catheter 105.

According to an example embodiment, an algorithm may be provided to calculate the position of the catheter in the camera's reference system (camera space). In this regard, to be able to visualize the catheter in the HMD, a way to transform from the camera space to the HMD's reference system (HMD space) may be required. According to an example embodiment, this may be accomplished by performing a one-time calibration with the calculated catheter position in the camera space and the virtual catheter position in the HMD space.

As noted above, the one-time calibration may be performed with the calculated catheter position in the camera space and the virtual catheter position in the display space. In an example embodiment, once calibrated, the entire position of the catheter may be calculated provided the makers are sufficiently detectable by the sensing unit 115.

In an example embodiment, the calibration process may include two steps. First, the user may use voice commands and gestures to move the virtual catheter to overlay the real one seen through the HMD. Then the user may issue a command, and the system may calculate T_{cam→hmd}. This transformation may be used for the remaining visualization session, and may be saved to be used for later sessions as well, as long as the camera is stationary.

FIG. 4 illustrates a reference system mapping process, according to an example embodiment. In an example embodiment, the coordinate of the catheter in its own reference system (model space) may be represented as A^{M}. At a certain frame, T_{cam} and T_{hmd} may transform A^{M} into the camera space and the HMD space, respectively. According to an example embodiment, it may be possible to determine a transformation T_{cam→hmd} that will transform from the camera space to the HMD space. This may be accomplished with equation (1) as shown below.

T_{cam}T_{cam→hmd}A^{M}=T_{hmd}A^{M} (1)

In order for this to work with every A^{M}, equation (2) shown below may be applied.

T_{cam→hmd}=T_{cam}⁻¹T_{hmd} (2)

Then, with every frame where a new T_{cam} is calculated from the position of the catheter in the image, it may be possible to calculate a corresponding T_{hmd} and display the virtual catheter.

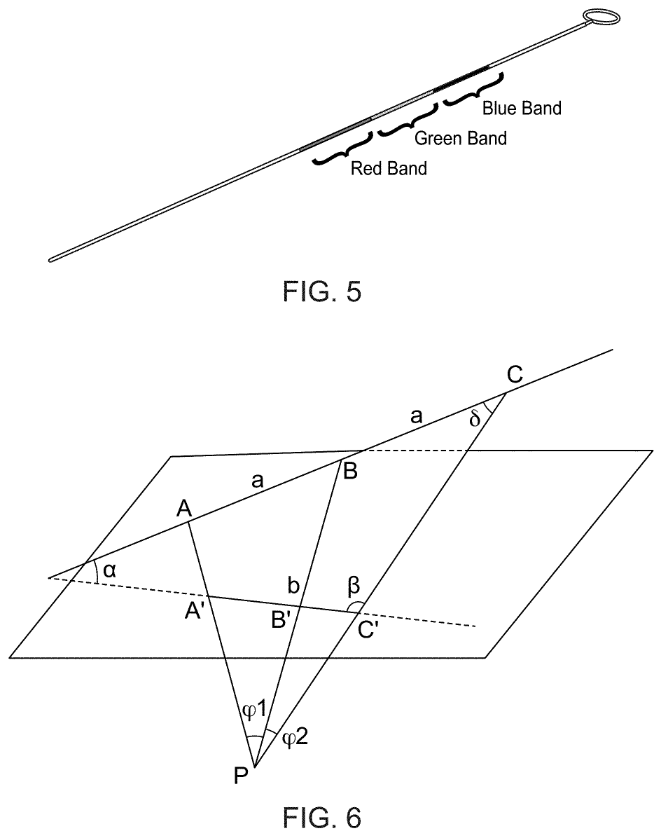

FIG. 5 illustrates a catheter with color bands, according to an example embodiment. As illustrated in FIG. 5, the catheter 105 may include three different color bands to provide a way of tracking the 3D position of the catheter in real-time. Various colors may be selected, however, FIG. 5 illustrates a catheter 105 of one embodiment that includes red, green, and blue color bands. According to one example embodiment, the color bands need to be distinct and continuous. The lengths of the three color bands, as well as the uncolored forward portion (from the catheter tip to the beginning of the first color band) of the catheter may be known by the person using the catheter. According to an example embodiment, the lengths of the color bands may be about 3.8 cm, and the uncolored portion may be about 12.1 cm. In certain example embodiments, the longer the color bands, the more accurate the system is. However, their combined length may not be longer than the length of the catheter. In addition, enough room should be reserved at the rear end of the catheter to allow holding by an operator or surgeon. In addition, the color bands may be adjacent to each other on the catheter. With the length and positions of the color band endpoints detected in the camera space, it may be possible to calculate the 5DOF (no roll) information of the catheter, and infer the position of the tip of the catheter.

FIG. 6 illustrates a special representation of the catheter on an image plane, according to an example embodiment. As illustrated in FIG. 6, a pinhole model may be used to represent the camera at point P, which represents the center of projection of the pinhole model, and is the origin point in the camera space. From FIG. 6, it may be possible to observe in the image, 2D coordinates of the catheter A′, B′, and C′. For instance, FIG. 6 illustrates three endpoints of two color bands as an example. Given the knowledge of the lengths of the color bands |AB| and |BC|, this may become a simplified version of the perspective three-point problem (P3P).

In a general P3P problem, there may be as many as four solutions. According to certain example embodiments, where the three points are collinear, there may be two solutions, of which only one may be desired. Thus, in certain example embodiments, determining the correct solution may require finding the angle between the catheter and its image in the image plane, as illustrated in FIG. 6. In addition, certain example embodiments may use a geometric method to find a, and the position of the catheter may be calculated as described herein. Further, the segment endpoints A′, B′, and C′ may be extracted from the image, according to an example embodiment.

According to an example embodiment, to find a, the three endpoints of two consecutive color bands may be denoted as A, B, and C in FIG. 6. As also illustrated in FIG. 6, the point P serves as the center of projection. The images of A, B, and C are A′, B′, and C′ on the image plane. Given the camera intrinsics which may be obtained by a one-time calibration, P, A′, B′, and C′ may be explicitly represented with exact coordinates in pixels. According to an example embodiment, the angles may be calculated according to equations (3) and (4) shown below.

$$\begin{matrix}∠ APB = {ϕ}_{1} = a cos \frac{{PA}^{′} \cdot {PB}^{′}}{\vert {PA}^{′} \vert \vert {PB}^{′} \vert} & ( 3 ) \\ ∠ BPC = {ϕ}_{2} = a cos \frac{{PB}^{′} \cdot {PC}^{′}}{\vert {PB}^{′} \vert \vert {PC}^{′} \vert} & ( 4 )\end{matrix}$$

With equations (3) and (4) in mind, it may be assumed that the two color segments are of equal length a (Assume |AB|=|BC|=a) and |PB|=b. Trigonometric equations give the following equations (5) and (6).

$$\begin{matrix}\frac{a}{sin {ϕ}_{1}} = \frac{b}{sin ( π - {ϕ}_{1} - {ϕ}_{2} - δ )} & ( 5 ) \\ \frac{a}{sin {ϕ}_{2}} = \frac{b}{sin δ} & ( 6 )\end{matrix}$$

δ is the angle ∠ACP, and every value in the above equations may be known. As such, it may be possible to solve for δ with equation (7) shown below.

$$δ = arctan ( \frac{sin {ϕ}_{2} sin ( {ϕ}_{1} + {ϕ}_{2} )}{sin {ϕ}_{1} - sin {ϕ}_{2} cos ( {ϕ}_{1} + {ϕ}_{2} )} ) \qquad ( 7 )$$

Once δ is solved, angle α may be determined with α=π−β−δ. Here, α may be either positive or negative depending on whether A is farther away from the image plane than C or closer. When A is closer than C (FIG. 6), α>0. However, when A is farther, α<0.

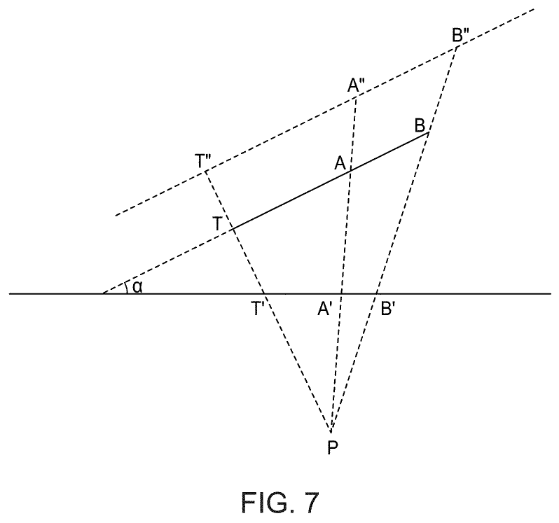

FIG. 7 illustrates a position of the catheter in a camera space, according to an example embodiment. With the 3D orientation obtained above, it may be possible to calculate the position of the catheter in the camera space. As illustrated in FIG. 7, P may represent the center of projection, TA may represent the uncolored forward portion of the catheter, and AB may represent the first color band on the catheter. FIG. 7 also illustrates AB, which may represent a color band, and AT may represent the portion of the catheter from the tip to the color band. In an example embodiment, AT may not be entirely visible from the camera. In addition, A′, B′, and T′ may represent the images of A, B, and T on the image plane, respectively. With respect to FIG. 7, the lengths of A′B′ are known since they were detected in the image. Furthermore, the lengths PA′ and PB′ are known.

In an example embodiment, the actual catheter may be in the plane formed by P and T′B′, and the orientation. However, it may be on any line that is parallel to TB, for example T″B″. According to an example embodiment, PA″ may be set to PA″=mPA′. The actual ratio m may not necessarily be significant in certain example embodiments since a different ratio only leads to a parallel line to BC. However, it may be assumed, for example, that m=10, and PB″=kPB′. Then, A″B″=PB″−PA″=kPB′−PA″.

According to an example embodiment, the angle formed by the catheter and its image a may be known, which may be the angle formed by A″B″ and A′B′.

A″B″·A′B′=|A″B″∥A′B′|cos α (8)

Equation (8) may further be transformed to equation (9) shown below.

kPB′·A′B′−PA″·A′B′=\sqrt{k²PB′²−2kPB′·PA″+PA″²}|A′B′|cos α (9)

Here, all values in equation (9) may be known except for k, where k is the ratio of PB″ over PB′. The value of k may be solved, making it possible to find the position of B^{∥}. In addition, according to an example embodiment, equation (9) may be a quadratic equation, and there may be two solutions for k. Judging from FIG. 7, if α>0, B may be further away from the image plane than A. Therefore, k may be greater than the assumed ratio of |PA″| over |PA′|, and vice versa.

Since the length of the catheter color band AB in the real world is known, it may be possible to calculate the position of the color band AB in the camera space with equations (10) and (11) shown below.

$$\begin{matrix}PA = \frac{\vert AB \vert}{\vert {A}^{″} {B}^{″} \vert} {PA}^{″} & ( 10 ) \\ PB = \frac{\vert PA \vert}{\vert {PA}^{″} \vert} {PB}^{″} & ( 11 )\end{matrix}$$

With equations (10) and (11), it may be possible to calculate the position of the catheter (i.e., position of the catheter tip) in the camera space. For example, this may be done with equation (12) shown below.

$$PT = PA - \frac{\vert AB \vert}{\vert AT \vert} AB \qquad ( 12 )$$

With equations (10), (11), and (12) it may be possible to calculate the position of the tip of the catheter in the camera space. This may also be made possible since the lengths of the uncolored forward portion of the catheter and the color bands are known. In an example embodiment, it may also be possible to calculate the position of the catheter with two adjacent color bands. The three color bands may be used to make the system robust against occlusion and improve accuracy when all three are visible.

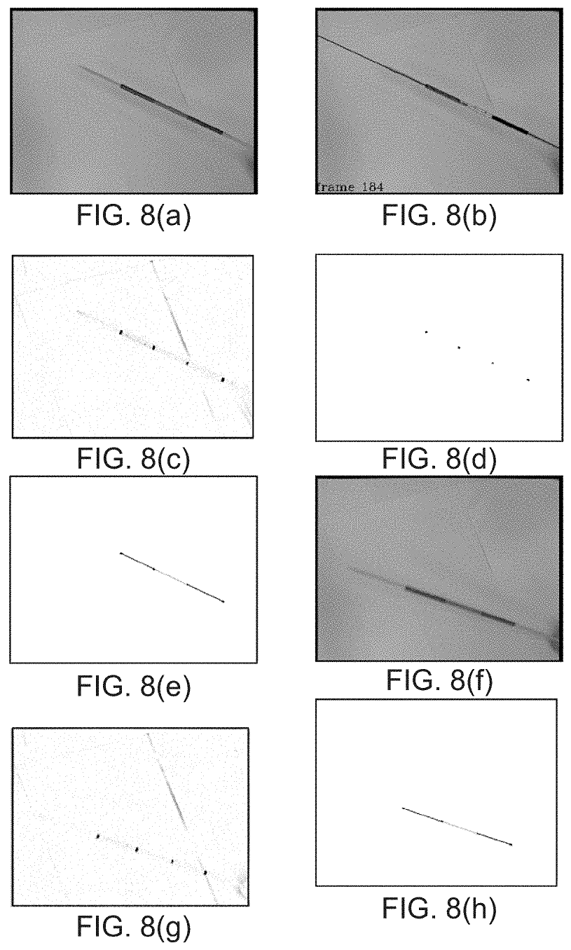

FIG. 8 (a)-8(h) illustrate a procedure for detecting endpoints of the color bands, according to an example embodiment. In an example embodiment, in the color band detection step, the input may be an image I of the catheter (P_{catheter} being all the pixels that belong to the catheter), and the output may be the locations of the endpoints of the color bands. In addition, the accuracy of the color band detection may be the most important factor in finding the catheter tip. That is, to reliably detect the endpoints of the color bands, the algorithm illustrated in FIG. 2 may be provided.

Beginning from a rectified image (FIG. 8(a)), simple thresholds maybe used to get the most of the pixels P_{catheter}*⊆P_{catheter} of the catheter in the image. The pixels may then be fit to a line, which may be called the axis Lₐₓᵢₛ of the catheter (FIG. 8(b)). Further, as illustrated in FIG. 8(c), the gradient G_{L}_{ₐₓᵢₛ} along the direction of Lₐₓᵢₛ may be calculated using a variation of the Sobel filter K_{L}_{ₐₓᵢₛ} that is weighed anisotropically according to Lₐₓᵢₛ. In addition, G_{L}_{ₐₓᵢₛ} may have high values near the border of two different colors. As illustrated in FIG. 8(d), after smoothing using a median filter, the gradient map may be thresholded into binary images G_{L}_{ₐₓᵢₛ}* with positive values where the borders are. Thresholded may refer to the pixels on the gradient map with a gradient value larger than or equal to a predefined threshold is set to have the value 1 in the binary image. In addition, the pixels on the gradient map with a gradient value smaller than the predefined threshold may be set to have value 0 in the binary image. Finally, as illustrated in FIG. 8(e), the weighted center of the connected components of G_{L}_{ₐₓᵢₛ} as the endpoints of color bands. Furthermore, as illustrated in FIGS. 8(f, g, h), the processing technique may be robust against blurry images caused by motion.

According to certain example embodiments, it may be possible to analyze the performance of the system. For example, this may be done by measuring the stability of the tracking algorithm. Then, the tracking accuracy may be tested on a grid, and experiments may be conducted in the physical world by moving the catheter, and comparing the calculated catheter tip location with ground truth from a third-party external tracker. The ground truth may refer to the 3D position of the catheter tip as well as the 3D orientation of the catheter produced by an established third-party external tracker.

Given the color segment endpoints detected in the image, the algorithm according to certain example embodiments may output the computed catheter position. The instability in the tracking algorithm may result from random noise in each frame. The noise may cause the same endpoint in two frames to be detected a few pixels apart, even when the catheter remains still.

In view of the potential instability that may result, certain example embodiments provide a way of measuring the stability as the root mean square of the change of calculated tip in the camera space in two consecutive frames, while keeping the catheter still. Given n frames, and the tip of the catheter in frame i as xᵢ, the stability may be measured as equation (13) shown below.

$${S}_{RMS} = \sqrt{\frac{1}{n - 1} {\sum }_{i = 1}^{n - 1} {‖ {x}_{i} - {x}_{i + 1} ‖}^{2}} \qquad ( 13 )$$

In certain example embodiments, several factors may influence the stability of the tracking algorithm, including lighting condition, threshold for color segmentation, and distance from the camera to the catheter. According to certain example embodiments, the algorithm may achieve a stability of 0.33 mm as measured over 870 frames.

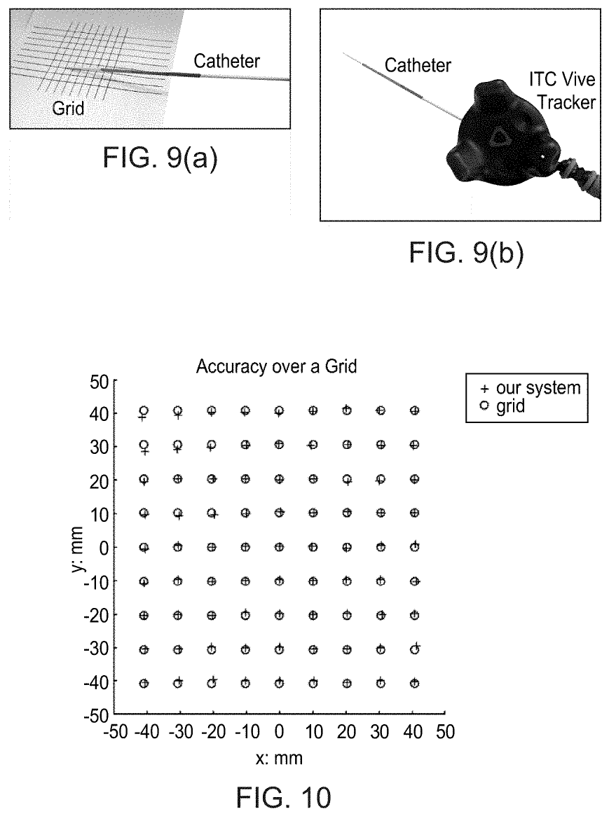

FIG. 9(a) illustrates a setup for testing the tracking accuracy over a grid, according to an example embodiment. According to certain example embodiments, to test the accuracy of the tracking algorithm, the tip of the catheter may be moved and pointed at the intersections of a grid. The grid, according to an example embodiment, may be about 8.16 cm×8.16 cm in size, and may be printed on a white sheet. This may provide enough space to test the accuracy of the catheter movement. Moreover, in an example embodiment, the catheter does not penetrate the skull by more than 6.5 cm, and the setup for this test is illustrated in FIG. 9(a). Before the actual measurements, the axis may be aligned with the grid along with a predefined marker sheet, and point the tip of the catheter to the center of the grid. In addition, when moving along the grid, the catheter's orientation may be kept fixed.

FIG. 10 illustrates an accuracy tracking approach over the grid, according to an example embodiment. In particular, FIG. 10 illustrates the catheter tip locations on the grid. The circles in FIG. 10 indicate the grid intersection, and the crosses indicate the computed catheter tip position. In certain example embodiments, the average distance from the catheter tip to the corresponding grid intersections may be about 0.58 mm.

FIG. 9(b) illustrates a setup for testing tracking accuracy with a third party tracker, according to an example embodiment. As illustrated in FIG. 9(b), the accuracy of the algorithm of certain example embodiments may be demonstrated when the catheter is moving in vertical space, and when its orientation changes. This may be done by attaching the catheter to a third-party positional tracker such as, for example, an HTCVive tracker. The setup for this test is illustrated in FIG. 9(b).

According to certain example embodiments, the test may run similarly to visualization. For example, a one-time calibration may be done to find the relation between the camera space and the tracker's tracking system (Vive space). In this case, A^{M} may represent the catheter's coordinates in its own model space, and T_{cam} may transform A^{M} into the camera space. In addition, Tᵥᵢᵥₑ may transform A^{M} into the camera space. Further Tᵥᵢᵥₑ may transform A^{M} vive space and transformation T_{vive→cam}TᵥᵢᵥₑA^{M} may be found.

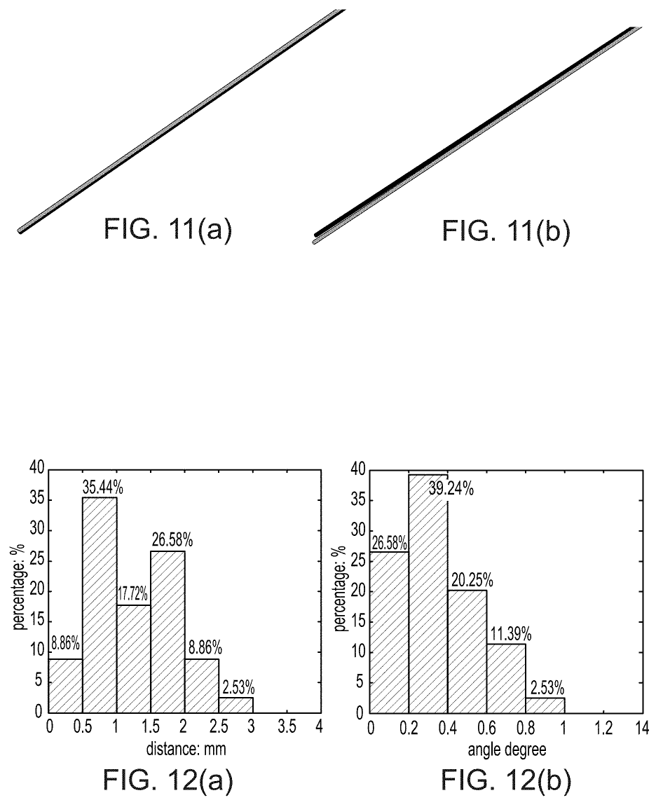

FIG. 11(a) illustrates the catheter from a camera and the third party tracker just after calibration with a calculated catheter position in a camera space and a virtual catheter position in the head-mounted display, according to an example embodiment. Further, FIG. 11(b) illustrates the catheter from a camera and the third party tracker with an unsteady alignment, according to an example embodiment. In an example embodiment for the first frame, the catheter may be racked by the camera and the tracker, and T_{vive→cam} may be calculated. For subsequent frames, the location of the catheter tip may be calculated in the camera space, both from the tracking algorithm of certain embodiments described herein, and transformed from the tracker.

At the beginning after the calibration (FIG. 11(a)), the representations of the catheter may be well aligned. However, as the catheter is moved, the algorithm and the tracker may start to produce slightly different positions, as illustrated in FIG. 11(b). According to certain example embodiments, the distance between the tracker reported tip position and the computed tip position may be measured, and the difference between the orientations of the catheter may be compared. In certain example embodiments, a total of 79 samples may be collected (after discarding one apparent invalid sample). The positions and orientations of the catheter may be recorded when they are not moving since there may be a lag between when the tracker updates its position and when the algorithm described above outputs the result.

FIG. 12(a) illustrates a distribution of the distances between the catheter's tip location and the third party tracker, according to an example embodiment. Further, FIG. 12(b) illustrates another distribution of an angle formed by the catheter orientations and the third party tracker, according to an example embodiment. In the 79 samples, the mean location was determined to have a mean of about 1.24 mm and a standard deviation of about 0.59 mm, and the orientation was determined to have a mean of about 0.36° and a standard deviation of about 0.20°.

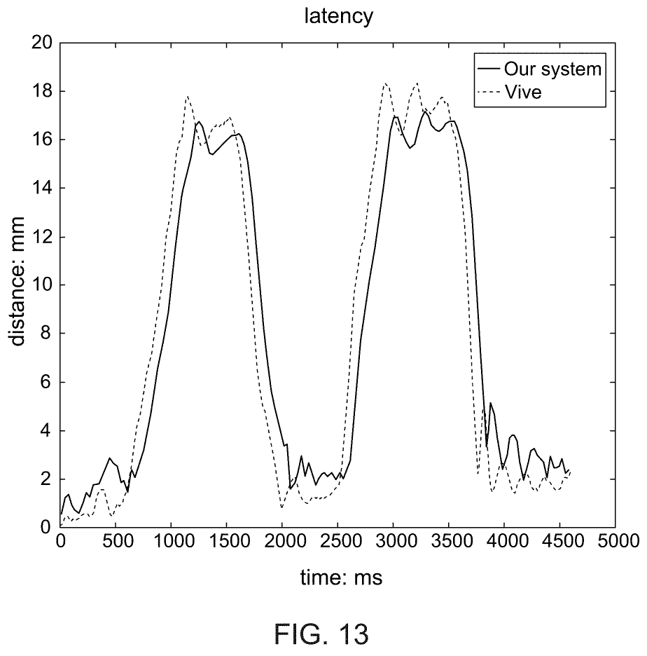

FIG. 13 illustrates a plot of recorded distances, according to an example embodiment. According to certain example embodiments, latency of the system may be measured and compared to the tracker. This latency may be measured as the time elapsed between the tracker update and the system catheter's location update. In certain example embodiments, the catheter may be moved rapidly, which is attached to the tracker, and recorded for each frame the distance from the catheter tip (tracked with the system and by the tracker) to their original location recorded at the beginning of the test. The distances may then be plotted, and the average time by which the system lags the tracker may be measured. As illustrated in FIG. 13, manual measurements show an average latency of about 95 ms. Further, Table 1 below illustrates a time breakdown of functions in tracking.

TABLE 1

| | | Avg. time per | |
|---|---|---|---|
| No. | Function | frame (ms) | % in processing |
| 1 | Undistort | 10.04 | 44.42 |
| 2 | Color Segmentation | 1.05 | 4.64 |
| 3 | Erosion | 0.23 | 1.01 |
| 4 | Get Axis | 0.93 | 4.13 |
| 5 | Gradient | 6.81 | 30.12 |
| 6 | Connected Components | 1.15 | 5.08 |
| 7 | Weighted Centroids | 0.92 | 4.09 |
| 8 | Calculate Catheter Position | 0.21 | 0.92 |

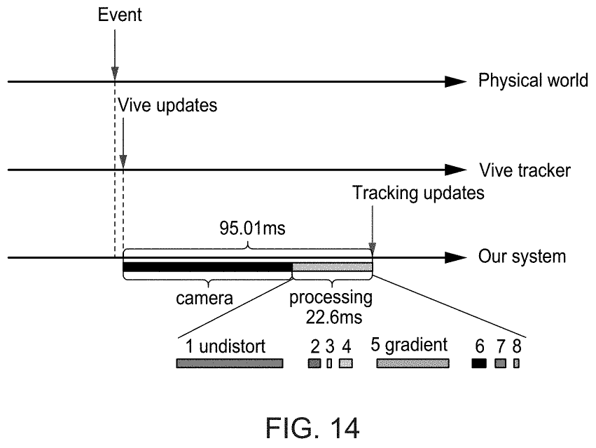

FIG. 14 illustrates various components of the system, according to an example embodiment. In the latency measurements, there may be two factors in the latency with respect to the tracker. For example, it may take about 72 ms for the camera (together with the driver and openCV functions) to capture and store the image. The processing may take an additional 22.6 ms in the machine of certain example embodiments. As illustrated in FIG. 14, undistorting may take the longest time, about 10.05 ms per frame (44.42% of the total processing time). The next most time-consuming task is calculating the gradient along the catheter axis, which may be about 6.1 ms per frame, 30.12% of the total processing time. The implementations in certain example embodiments may be performed on the CPU. In certain example embodiments, a GPU may reduce the processing time through parallelization, and a professional-grade camera with a low image capture time may reduce the latency.

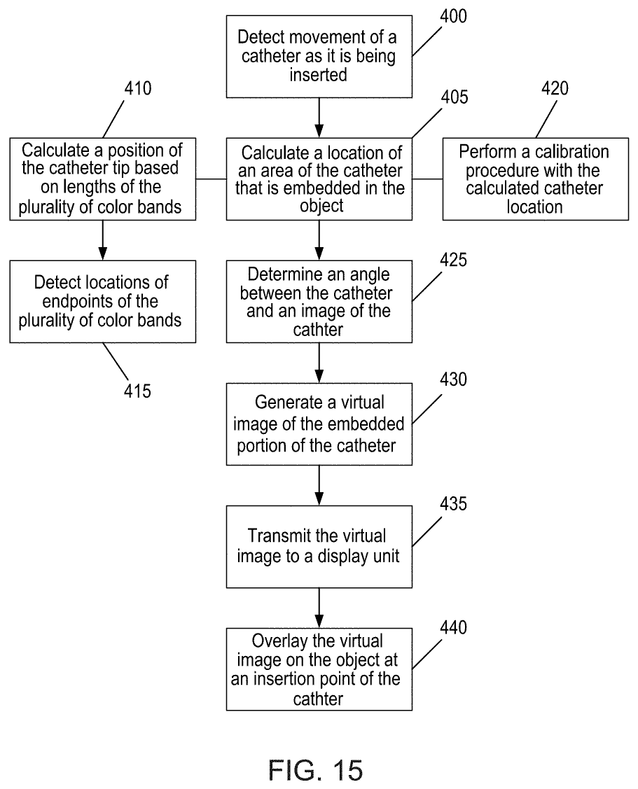

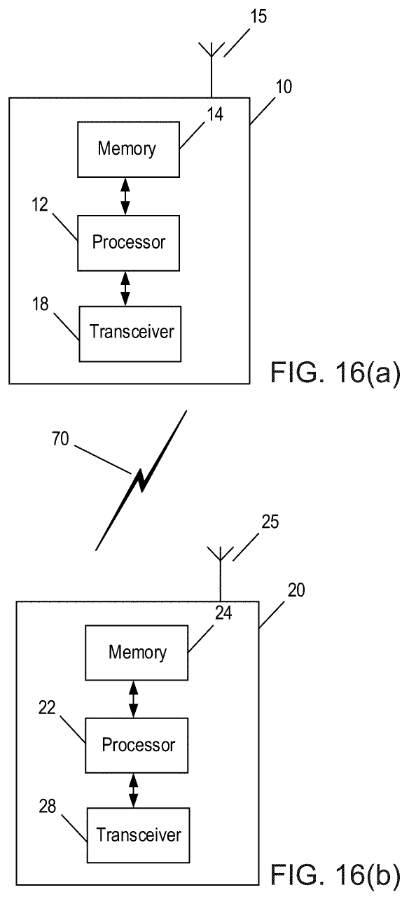

FIG. 15 illustrates a flow diagram of a method for visualizing and tracking a catheter, according to an example embodiment. In certain example embodiments, the flow diagram of FIG. 15 may be performed by a processing unit, such as processing unit 10 illustrated in FIG. 16(a). According to one example embodiment, the method of FIG. 15 may include initially at 400, detecting movement of a catheter as it is being inserted into an object. In an example embodiment, the object may include a human patient. The method may further include, at 405, calculating a location of an area of the catheter that is embedded in the object. According to an example embodiment, at 410, the calculating may include calculating a position of a tip of the catheter based on lengths of the plurality of color bands on the catheter. According to another example embodiment, at 415, the calculating may include detecting locations of endpoints of the plurality of color bands on the catheter.

In an example embodiment, detecting movement of the catheter may be performed by a sensing unit. In another example embodiment, at 420, the method may include performing a calibration procedure with the calculated catheter location in a sensing unit space and a virtual catheter position in a display space of the display unit. According to an example embodiment, at 425, the calculating may include determining an angle between the catheter and an image of the catheter in an image plane. At 430, the method may include generating a virtual image of the embedded portion of the catheter based on the detected movement of the catheter and location of the area of the catheter that is embedded. At 435, the method may include transmitting the virtual image of the embedded area of the catheter to a display unit. In addition, at 440, the method may include overlaying the virtual image in a user's field of view in the display unit to mimic the position of the entire catheter including the area embedded in the object.

According to an example embodiment, the catheter may include a plurality of tracking markers. According to another example embodiment, the plurality of tracking markers may include a plurality of color bands that are adjacent to each other.

FIG. 16(a) illustrates an example apparatus 10 according to an example embodiment. In an example embodiment, apparatus 10 may be a processing unit in a system such as for example, an augmented reality guided catheter system.

In some example embodiments, apparatus 10 may include one or more processors, one or more computer-readable storage medium (for example, memory, storage, or the like), one or more radio access components (for example, a modem, a transceiver, or the like), and/or a user interface. In an example embodiment, apparatus 10 may include a server, computer, or other device capable of executing arithmetic, logical operations, or control operations including for example, system control operations of one or a plurality of devices of the system. It should be noted that one of ordinary skill in the art would understand that apparatus 10 may include components or features not shown in FIG. 16(a).

As illustrated in the example of FIG. 16(a), apparatus 10 may include a processor 12 for processing information and executing instructions or operations. Processor 12 may be any type of general or specific purpose processor. In fact, processor 12 may include one or more of general-purpose computers, special purpose computers, microprocessors, digital signal processors (DSPs), field-programmable gate arrays (FPGAs), application-specific integrated circuits (ASICs), and processors based on a multi-core processor architecture, as examples. In further example embodiments, processor 12 may include a specialized processor or a ML/data analytics based application processor, such as a graphics processing unit (GPU) or tensor processing unit (TPU). In yet a further example, processor 12 may include a neural network or long short term memory (LSTM) architecture or hardware, etc.

While a single processor 12 is shown in FIG. 16(a), multiple processors may be utilized according to other example embodiments. For example, it should be understood that, in certain example embodiments, apparatus 10 may include two or more processors that may form a multiprocessor system (e.g., in this case processor 12 may represent a multiprocessor) that may support multiprocessing. In certain example embodiments, the multiprocessor system may be tightly coupled or loosely coupled (e.g., to form a computer cluster.

Processor 12 may perform functions associated with the operation of apparatus 10, which may include, for example, executing the process illustrated in the example of FIGS. 1-15.

Apparatus 10 may further include or be coupled to a memory 14 (internal or external), which may be coupled to processor 12, for storing information and instructions that may be executed by processor 12. Memory 14 may be one or more memories and of any type suitable to the local application environment, and may be implemented using any suitable volatile or nonvolatile data storage technology such as a semiconductor-based memory device, a magnetic memory device and system, an optical memory device and system, fixed memory, and/or removable memory. For example, memory 14 can be comprised of any combination of random access memory (RAM), read only memory (ROM), static storage such as a magnetic or optical disk, hard disk drive (HDD), or any other type of non-transitory machine or computer readable media. The instructions stored in memory 14 may include program instructions or computer program code that, when executed by processor 12, enable the apparatus 10 to perform tasks as described herein.

In an example embodiment, apparatus 10 may further include or be coupled to (internal or external) a drive or port that is configured to accept and read an external computer readable storage medium, such as an optical disc, USB drive, flash drive, or any other storage medium. For example, the external computer readable storage medium may store a computer program or software for execution by processor 12 and/or apparatus 10.

In some example embodiments, apparatus 10 may further include or be coupled to a transceiver 18 configured to transmit and receive information. Additionally or alternatively, in some example embodiments, apparatus 10 may include an input and/or output device (I/O device).

In an example embodiment, memory 14 may store software modules that provide functionality when executed by processor 12. The modules may include, for example, an operating system that provides operating system functionality for apparatus 10. The memory may also store one or more functional modules, such as an application or program, to provide additional functionality for apparatus 10. The components of apparatus 10 may be implemented in hardware, or as any suitable combination of hardware and software. According to an example embodiment, apparatus 10 may optionally be configured to communicate with apparatus 20 via a wireless or wired communications link 70 according various technologies including, for example, Wi-Fi or Bluetooth®.

According to some example embodiments, processor 12 and memory 14 may be included in or may form a part of processing circuitry or control circuitry. In addition, in some example embodiments, transceiver 18 may be included in or may form a part of transceiving circuitry.

According to example embodiments, apparatus 10 may be controlled by memory 14 and processor 12 to perform the functions associated with any of the example embodiments described herein, such as the system or signaling flow diagrams illustrated in FIGS. 1-15.

For instance, in one example embodiment, apparatus 10 may be controlled by memory 14 and processor 12 to detect movement of a catheter as it is being inserted into an object. The apparatus 10 may also be controlled by memory 14 and processor 12 to calculate a location of an area of the catheter that is embedded in the object. In addition, the apparatus 10 may also be controlled by memory 14 and processor 12 to generate a virtual image of the embedded portion of the catheter based on the detected movement of the catheter and the location of the area of the catheter that is embedded. Further, apparatus 10 may also be controlled by memory 14 and processor 12 to transmit the virtual image of the embedded area of the catheter to a display unit. The apparatus 10 may also be controlled by memory 14 and processor 12 to overlay the virtual image in a user's field of view in the display unit to mimic the position of the entire catheter including the area embedded in the object.

In another example embodiment, the apparatus 10 may be controlled by memory 14 and processor 12 to perform a calibration procedure with the calculated catheter location in a sensing unit space and a virtual catheter position in a display space of the display unit. According to an example embodiment, the catheter may include a plurality of tracking makers. In an example embodiment, the plurality of tracking markers may include a plurality of color bands that are adjacent to each other. In another example embodiment, the calculation may include calculating a position of a tip of the catheter based on lengths of the plurality of color bands. In a further example embodiment, the calculation may include detecting locations of endpoints of the plurality of color bands, and include determining an angle between the catheter and an image of the catheter in an image plane. In a further example embodiment, detecting movement of the catheter may be performed by a sensing unit.

FIG. 16(b) illustrates an example of an apparatus 20 according to one example embodiment. In an example embodiment, apparatus 20 may include a sensor device or unit, or a display unit. For example, the apparatus 20 may be a camera, a head-mounted display (HMD), an external monitor, or a projection that is viewable by an operator. It should be noted that one of ordinary skill in the art would understand that apparatus 20 may include components or features not shown in FIG. 16(b).

As illustrated in the example of FIG. 16(b), apparatus 20 may include a processor 22 for processing information and executing instructions or operations. Processor 22 may be any type of general or specific purpose processor. In fact, processor 22 may include one or more of general-purpose computers, special purpose computers, microprocessors, digital signal processors (DSPs), field-programmable gate arrays (FPGAs), application-specific integrated circuits (ASICs), and processors based on a multi-core processor architecture, as examples. In further example embodiments, processor 22 may include a specialized processor or a ML/data analytics based application processor, such as a graphics processing unit (GPU) or tensor processing unit (TPU). In yet a further example, processor 22 may include a neural network or long short term memory (LSTM) architecture or hardware, etc.

While a single processor 22 is shown in FIG. 16(b), multiple processors may be utilized according to other example embodiments. For example, it should be understood that, in certain example embodiments, apparatus 20 may include two or more processors that may form a multiprocessor system (e.g., in this case processor 22 may represent a multiprocessor) that may support multiprocessing. In certain example embodiments, the multiprocessor system may be tightly coupled or loosely coupled (e.g., to form a computer cluster).

Processor 22 may perform functions associated with the operation of apparatus 20, which may include, for example, executing the process illustrated in the example of FIGS. 1-15.

Apparatus 20 may further include or be coupled to a memory 24 (internal or external), which may be coupled to processor 22, for storing information and instructions that may be executed by processor 22. Memory 24 may be one or more memories and of any type suitable to the local application environment, and may be implemented using any suitable volatile or nonvolatile data storage technology such as a semiconductor-based memory device, a magnetic memory device and system, an optical memory device and system, fixed memory, and/or removable memory. For example, memory 24 can be comprised of any combination of random access memory (RAM), read only memory (ROM), static storage such as a magnetic or optical disk, hard disk drive (HDD), or any other type of non-transitory machine or computer readable media. The instructions stored in memory 24 may include program instructions or computer program code that, when executed by processor 22, enable the apparatus 20 to perform tasks as described herein.

In an example embodiment, apparatus 20 may further include or be coupled to (internal or external) a drive or port that is configured to accept and read an external computer readable storage medium, such as an optical disc, USB drive, flash drive, or any other storage medium. For example, the external computer readable storage medium may store a computer program or software for execution by processor 22 and/or apparatus 20.

In some example embodiments, apparatus 20 may further include or be coupled to a transceiver 28 configured to transmit and receive information. Additionally or alternatively, in some example embodiments, apparatus 20 may include an input and/or output device (I/O device).

In an example embodiment, memory 24 may store software modules that provide functionality when executed by processor 22. The modules may include, for example, an operating system that provides operating system functionality for apparatus 20. The memory may also store one or more functional modules, such as an application or program, to provide additional functionality for apparatus 20. The components of apparatus 20 may be implemented in hardware, or as any suitable combination of hardware and software.

According to some example embodiments, processor 22 and memory 24 may be included in or may form a part of processing circuitry or control circuitry. In addition, in some example embodiments, transceiver 18 may be included in or may form a part of transceiving circuitry.

According to example embodiments, apparatus 20 may be controlled by memory 24 and processor 22 to perform the functions associated with any of the example embodiments described herein, such as the system or signaling flow diagrams illustrated in FIGS. 1-15.

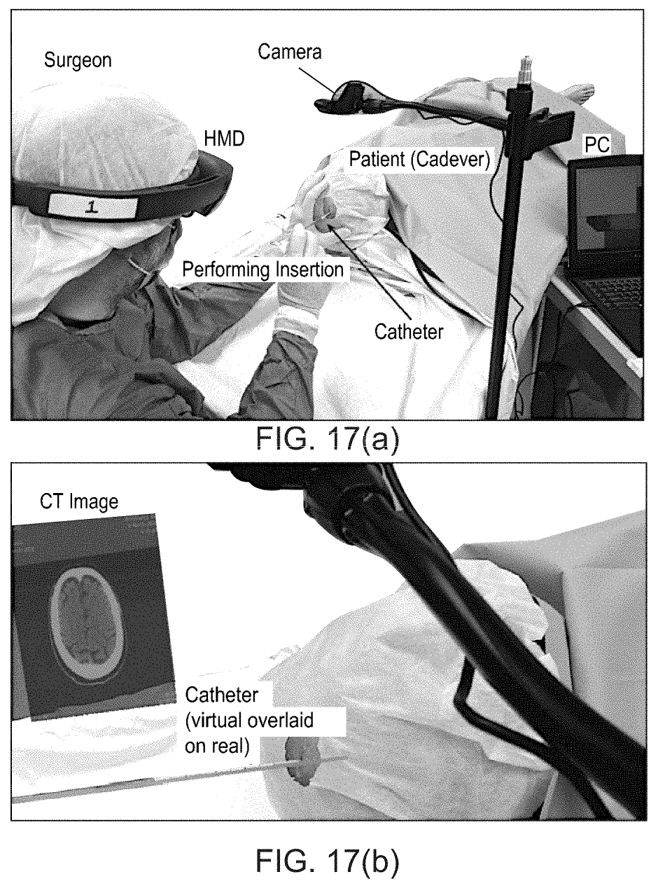

Certain example embodiments provide several technical improvements, enhancements, and/or advantages. Various example embodiments may, for example, provide a system that provides an optical marker and tracking technique, suitable for augmented reality. Certain example embodiments may also make use of a catheter with minimal changes to the shape and/or weight of the catheter, and provide an algorithm to detect the color bands on the catheter and use them to calculate the position of the catheter. Certain example embodiments further provide the ability to know and visualize a tip of a needed that is occluded, as long as enough of the colored portion remains visible. Other example embodiments may only need a one-time calibration to determine the relation between the HMD and the camera, and be able to achieve high accuracy and low latency. Moreover, as illustrated in FIGS. 17(a) and 17(b), certain example embodiments may have useful applications in surgical environments.

According to further example embodiments, using a stationary camera for tracking the catheter eliminates the requirement of the user of the HMD to be looking at the catheter to track it. This therefore may allow an operator such as a medical personnel (e.g., doctor, surgeon, etc.) to freely look anywhere without losing the tracking of the catheter. In addition, according to other example embodiments, the system described herein is not tied to the HMD, and may be capable of process images from the camera, as well as the medical volume separately on another machine. Such example embodiments make it possible to achieve faster and more accurate sensing and higher fidelity medical images.

In additional example embodiments, it may be possible to calculate the position of the catheter with two adjacent color bands, and improve the robustness and accuracy of the system by using three color bands when all three color bands are visible. Further, compared to existing systems, certain example embodiments may provide a low-latency, high-performance way to track catheters and other 5DOF thin cylindrical objects. Other example embodiments may also provide an image processing algorithm to extract tracking color segment endpoints in an image, and perform tests in which the catheter is moved over a grid to show that it is possible to achieve a 0.58 mm accuracy. According to certain example embodiments, processing for each frame may take about 22.6 ms on a moderately powerful computer. Moreover, the color markers and tracking technique in certain example embodiments may be applied to other catheterization procedures, or other areas where SDOF tracking is required.

In some example embodiments, the functionality of any of the methods, processes, signaling diagrams, algorithms or flow charts described herein may be implemented by software and/or computer program code or portions of code stored in memory or other computer readable or tangible media, and executed by a processor.

In some example embodiments, an apparatus may be included or be associated with at least one software application, module, unit or entity configured as arithmetic operation(s), or as a program or portions of it (including an added or updated software routine), executed by at least one operation processor. Programs, also called program products or computer programs, including software routines, applets and macros, may be stored in any apparatus-readable data storage medium and include program instructions to perform particular tasks.

A computer program product may comprise one or more computer-executable components which, when the program is run, are configured to carry out some of the various example embodiments described herein. The one or more computer-executable components may be at least one software code or portions of it. Modifications and configurations required for implementing functionality of an example embodiment may be performed as routine(s), which may be implemented as added or updated software routine(s). Software routine(s) may be downloaded into the apparatus.

As an example, software or a computer program code or portions of it may be in a source code form, object code form, or in some intermediate form, and it may be stored in some sort of carrier, distribution medium, or computer readable medium, which may be any entity or device capable of carrying the program. Such carriers may include a record medium, computer memory, read-only memory, photoelectrical and/or electrical carrier signal, telecommunications signal, and software distribution package, for example. Depending on the processing power needed, the computer program may be executed in a single electronic digital computer or it may be distributed amongst a number of computers. The computer readable medium or computer readable storage medium may be a non-transitory medium.

In other example embodiments, the functionality may be performed by hardware or circuitry included in an apparatus, for example through the use of an application specific integrated circuit (ASIC), a programmable gate array (PGA), a field programmable gate array (FPGA), or any other combination of hardware and software. In yet another example embodiment, the functionality may be implemented as a signal, a non-tangible means that can be carried by an electromagnetic signal downloaded from the Internet or other network.

According to an example embodiment, an apparatus, such as a node, device, or a corresponding component, may be configured as circuitry, a computer or a microprocessor, such as single-chip computer element, or as a chipset, including at least a memory for providing storage capacity used for arithmetic operation and an operation processor for executing the arithmetic operation.

One having ordinary skill in the art will readily understand that the example embodiments as discussed above may be practiced with steps in a different order, and/or with hardware elements in configurations which are different than those which are disclosed. Therefore, although some embodiments have been described based upon these example preferred embodiments, it would be apparent to those of skill in the art that certain modifications, variations, and alternative constructions would be apparent, while remaining within the spirit and scope of example embodiments. In order to determine the metes and bounds of the example embodiments, therefore, reference should be made to the appended claims.

We claim:

1. A method for visualizing and tracking a catheter, comprising:
detecting movement of the catheter as it is being inserted into an object;
calculating a location of an area of the catheter that is embedded in the object;
generating a virtual image of the embedded portion of the catheter based on the detected movement of the catheter and location of the area of the catheter that is embedded;
transmitting the virtual image of the embedded area of the catheter to a display unit; and
overlaying the virtual image in a user's field of view in the display unit to mimic the position of the entire catheter including the area embedded in the object.

2. The method according to claim 1, wherein the catheter comprises a plurality of tracking markers.

3. The method according to claim 2, wherein the plurality of tracking markers comprises a plurality of color bands that are adjacent to each other.

4. The method according to claim 3, wherein the calculation further comprises calculating a position of a tip of the catheter based on lengths of the plurality of color bands.

5. The method according to claim 3, wherein the calculation further comprises detecting locations of endpoints of the plurality of color bands.

6. The method according to claim 1,
wherein detecting movement of the catheter is performed by a sensing unit, and
wherein the method further comprises performing a calibration procedure with the calculated catheter location in a sensing unit space and a virtual catheter position in a display space of the display unit.

7. The method according to claim 1, wherein the calculation further comprises determining an angle between the catheter and an image of the catheter in an image plane.

8. An apparatus for visualizing and tracking a catheter, the apparatus comprising:
at least one processor; and
at least one memory comprising computer program code,
the at least one memory and computer program code configured, with the at least one processor, to cause the apparatus at least to
detect movement of a catheter as it is being inserted into an object;
calculate a location of an area of the catheter that is embedded in the object;
generate a virtual image of the embedded portion of the catheter based on the detected movement of the catheter and location of the area of the catheter that is embedded;
transmit the virtual image of the embedded area of the catheter to a display unit; and
overlay the virtual image in a user's field of view in the display unit to mimic the position of the entire catheter including the area embedded in the object.

9. The apparatus according to claim 8, wherein the catheter comprises a plurality of tracking markers.

10. The apparatus according to claim 9, wherein the plurality of tracking markers comprises a plurality of color bands that are adjacent to each other.

11. The apparatus according to claim 10, wherein the calculation further comprises calculating a position of a tip of the catheter based on lengths of the plurality of color bands.

12. The apparatus according to claim 10, wherein the calculation further comprises detecting locations of endpoints of the plurality of color bands.

13. The apparatus according to claim 8,
wherein detecting movement of the catheter is performed by a sensing unit, and
wherein the at least one memory and computer program code are further configured, with the at least one processor, to cause the apparatus at least to perform a calibration procedure with the calculated catheter position in a sensing unit space and a virtual catheter position in a display space of the display unit.

14. The apparatus according to claim 8, wherein the calculation further comprises determining an angle between the catheter and an image of the catheter in an image plane.

15. A system for visualizing and tracking a catheter, comprising:
sensing means for detecting movement of a catheter as it is being inserted into an object;
processing means for calculating, based on information obtain from the sensing means, a location of an area of the catheter that is embedded in the object;
generating means for generating a virtual image of the embedded portion of the catheter based on the detected movement of the catheter and the location of the area of the area of the catheter that is embedded in the object;
transmitting means for transmitting the virtual image of the embedded area of the catheter to a display unit; and
displaying means for overlaying the virtual image in a user's field of view in the display unit to mimic the position of the entire catheter including the area embedded in the object.

16. The system according to claim 15, wherein the catheter comprises a plurality of tracking markers.

17. The system according to claim 16, wherein the plurality of tracking markers comprises a plurality of color bands that are adjacent to each other.

18. The system according to claim 17, wherein the processing means further comprises calculating a position of a tip of the catheter based on lengths of the plurality of color bands.

19. The system according to claim 17, wherein the processing means further comprises detecting locations of endpoints of the plurality of color bands.

20. A computer program, embodied on a non-transitory computer readable medium, the computer program, when executed by a processor, causes the processor to perform the method according to claim 1.
